(19) United States
(12) Patent Application Publication
(10) Pub. No.: US 2009/0125067 A1
(43) Pub. Date: May 14, 2009

(54) IN-LINE OCCIPITAL PLATE AND METHOD OF USE

(75) Inventors: Michael J. Mazzuca, North Easton, MA (US); Connie P. Marchek, Foxboro, MA (US); Michael D. Sorrenti, Middleboro, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DEPUY SPINE, INC., Raynham, MA (US)

(21) Appl. No.: 11/936,904

(22) Filed: Nov. 8, 2007

(51) Int. Cl. A61B 17/58; A61B 17/56

(52) U.S. Cl. 606/280; 606/246; 606/301

(57) ABSTRACT

Various embodiments of an implantable spinal fixation device are provided herein. In general, the device can include an elongate member having a first end and a second end having a center-line extending therebetween. Further, the elongate member can include any number of bone screw receiving thru-hole(s) positioned proximate (e.g., along or offset from) the center of the elongate member. Further, the device can include a position-adjustable coupling element proximate the thru-hole(s), and configured to releasably engage a spinal fixation element. Additionally, methods of occipital coupling of a spinal fixation element are provided herein.

### FIELD OF USE

The present disclosure relates to devices and methods for use in various spinal fixation procedures, and in particular to devices and methods for use in cervical stabilization procedures.

### BACKGROUND

Stabilization of the spine is often required following trauma, tumor, or degenerative pathologies. Although each region of the spine presents unique clinical challenges, posterior fixation of the cervical spine is particularly challenging because the anatomy of the cervical spine makes it a technically difficult area to instrument. Specifically, several vital neural and vascular structures, including the vertebral arteries, nerve roots, and spinal cord must be avoided during surgery.

Current methods of posterior cervical stabilization include the use of a mid-line occipital spinal plate and various fixation elements (e.g., fixation rods). The fixation elements are coupled to adjacent vertebrae by attachment to various anchoring devices, such as hooks, bolts, wires, or screws. Often, two rods are disposed on opposite sides of the spinous process in a substantially parallel relationship. The fixation elements can have a predetermined contour that has been designed according to the properties of the target implantation site, and once installed, the fixation elements hold the vertebrae in a desired spatial relationship, either until healing or spinal fusion has taken place, or for some longer period of time. When such surgery is performed in the cervical spine, the proximal ends of the rods are typically molded according to the anatomy of the skull and the cervical spine, and attached to a fixation plate that is implanted in the occiput.

Typically, a single occipital plate (e.g., a T-shaped or Y-shaped plate) is positioned along the midline of a patient's occipital bone so that the single plate can engage adjacent spinal fixation elements that run on either side of the midline. Thus, as opposed to selecting an optimal position (e.g., an area of high bone density) to engage the fixation plate to the occipital bone, the surgeon must select a position capable of accommodating both the first and second fixation elements. As an additional drawback, in use, it is often difficult to engage the fixation element(s) to such a fixation plate once the fixation plate is engaged to the desired anatomical location. In an attempt to overcome such difficulties, some procedures utilize a one-piece design (i.e., the fixation element engaged to the fixation plate prior to use). However, such devices can be difficult to use in that they can limit the surgeon's ability to select the optimal engagement point on the occipital bone and/or the vertebrae. As an additional problem, use of such mid-line plates can also be limited by the patient's anatomy. For example, some patients, either from a previous surgical procedure or from natural causes, have an enlarged foramen magnum thereby eliminating the possibility of using any type of mid-line fixation plate.

Thus, there remains a need for devices and methods capable of improving and/or optimizing cervical stabilization procedures.

### SUMMARY

Devices and methods for enhancing the effectiveness of spinal fixation surgery are provided herein. In general, the devices and methods described below provide a surgeon with the ability to optimize the selection of an engagement point for a spinal fixation element relative to a patient's occipital bone. In determining such an optimal location, the surgeon is now free to weigh variables such as bone thickness and/or bone density, size/shape of the patient's foramen magnum, etc. without the burden of selecting a location suitable for both first and second fixation elements (e.g., rods) and/or the exact orientation of the fixation element relative to the fixation plate. Thus, the devices and methods allow the surgeon to engage a fixation plate at an optimal location of the occipital bone, position a spinal fixation element along a series of vertebrae, manipulate a coupling element of the fixation plate so as to align the coupling element with the superior end of the fixation element, and securely engage the fixation element to the coupling element. As will be shown, this flexibility provides enhanced stability, effectiveness, and usefulness for such spinal stabilization procedures.

Various aspects of such a spinal fixation device are provided herein. In a first aspect, the device can include an elongate member having a first end and a second end with a center-line extending therebetween. As will be described, the center-line can be straight, curved, etc. Further, the device can include any number of bone screw receiving thru-hole(s) (e.g., 1, 2, 3, 4, etc.) formed in the elongate member thereby allowing the device to be secured to the desired anatomical location. In an exemplary embodiment, the thru-holes are positioned proximate the center-line of the elongate member. For example, the thru-holes can be positioned along the center line or at least one thru-hole can be positioned offset from the center line (e.g., the holes can be staggered along the center line). Additionally, the length and/or width of the elongate member can be configured to optimize the given procedure. The elongate member can also be formed of a wide range of biocompatible materials (e.g., various polymers, polymer blends, metals, etc.). In an exemplary embodiment, the elongate member can be configured to conform to the surface of a target anatomical location.

The device can further include a position-adjustable coupling element configured to releasably engage a spinal fixation element formed on or engaged to a location proximate (e.g., aligned with or off-set from) the center-line of the elongate member. In an exemplary embodiment, the coupling element is rotatable and is in alignment with the thru-hole(s). As will be described, the coupling element can be any element capable of releasably engaging a fixation element to the elongate member. For example, the coupling element can include a substantially “U-shaped” opening having a central channel configured to receive the spinal fixation element. In an exemplary embodiment, the coupling element can be a slotted bolt. The coupling element can be formed on and/or engaged to the elongate member in any number of manners. For example, the coupling element can be engaged to a thru-hole (e.g., an elongate thru-hole) in the elongate member. Adding to the versatility of the device, the coupling element can be positioned at various locations of the elongate member. For instance, the coupling element can be positioned substantially in the middle of the elongate member, at an inferior portion of the elongate member, etc. Thus, the coupling element can be formed on or engaged to the elongate member in any number of ways and at varying positions relative to the elongate member so as to optimize the efficiency and resulting stability of the fixation procedure.

As indicated above, the coupling element can be configured in various ways so as to facilitate engagement of a fixation element to the device. For example, in addition to being rotatable, the coupling element can be translatable and/or be capable of polyaxial movement relative to the elongate member. As will be described in detail below, such rotatable, translatable, and/or polyaxial movement of the coupling element relative to the elongate member can be provided in any number of ways.

In another aspect, an in-line occipital plate is provided which includes an elongate plate member with a center-line (straight or curved) extending from a first end of the member to a second end of the member wherein the elongate plate member is conformable to an anatomical location. Further, the elongate member can include a single position-adjustable coupling element configured to releasably engage a single spinal fixation element, and the member can further include at least one bone screw receiving thru-hole. In an exemplary embodiment, the rotatable coupling element and the bone screw receiving thru-hole(s) are positioned proximate the center-line of the elongate member. Similar to above, the coupling element can be translatable along the center-line of the elongate member. Also, in some embodiments, the coupling element can be configured for polyaxial movement relative to the elongate member.

In yet another aspect, an implantable spinal fixation device is provided which includes an occipital plate having a first end, a second end, and a center-line extending therebetween. Further, the occipital plate can include a plurality of bend zones to accommodate a location adjacent a midline of a patient's occipital bone. Also, similar to those embodiments summarized above, the spinal fixation device can include a plurality of thru-holes proximate the center-line of the occipital plate. In an exemplary embodiment, the occipital plate includes a single position adjustable (e.g., rotatable and/or translatable) coupling element positioned within an elongate thru-hole. For example, the coupling element can include a U-shaped opening having a central channel. Similar to above, the coupling element can also be configured for polyaxial movement relative to the elongate member.

Various aspects of a system of providing spinal stabilization are also provided. In one such aspect, the system includes an embodiment of a presently provided occipital plate connected to an occiput, a bone anchor (one or a plurality of such anchors) implanted in a vertebra(e), and a spinal fixation element connecting the bone anchor(s) and the occipital plate.

Additionally, various aspects of a method for occipital coupling of a spinal fixation element are also provided herein. In one such aspect, the method includes fixing an inferior portion of a spinal fixation element to one or more vertebrae. The method also includes providing an occipital plate having a superior end and an inferior end and a plurality of bone screw receiving thru-holes positioned proximate the center-line of the occipital plate. Additionally, the plate can include a position adjustable (e.g., rotatable) coupling element substantially aligned with (or offset from) the center-line of the occipital plate. The method further includes fixing the occipital plate to an anatomical location which is adjacent the foramen magnum and offset from an axis defined by the spinal column, and fixing a superior portion of the spinal fixation element to the rotatable coupling element of the plate. Optionally, the method can include coupling a second fixation element to a second occipital plate thereby allowing for first and second fixation elements to be positioned on opposite sides of the patient's spinal column.

Similar to the aspects described above, the method can also include various steps for manipulating the coupling element relative to the superior end of the fixation element so as to align the coupling element with the fixation element thereby facilitating fixation. For example, the method can include rotating, translating and/or polyaxially adjusting the coupling element relative to the occipital plate so as to align the coupling with the superior portion of the spinal fixation element.

Additionally, the method can include positioning first and second fixation elements on opposite sides of a patient's spinal column (e.g., along opposite sides of the midline of the spinal column). For example, the method can include fixing (e.g., via bone anchors) first and second spinal fixation elements to at least one vertebra. The method can also include providing first and second occipital plates. Like above, each occipital plate can have a first end, a second end and a center-line extending therebetween. The plates can also include a plurality of thru-holes positioned proximate the center-line, and a single position adjustable (e.g., rotatable and/or translatable) coupling element positioned within an elongate thru-hole. The method can further include fixing the first occipital plate adjacent the foramen magnum and offset in a first lateral direction from an axis defined by the spinal column, and fixing the second occipital plate adjacent the foramen magnum and offset in a second lateral direction from an axis defined by the spinal column. The method can further include manipulating the coupling element(s) relative to the first and second occipital plates so as to align each coupling element with the superior portion of a corresponding spinal fixation element. Once properly aligned, the method can include fixing a superior portion of the first and second spinal fixation elements to the first and second position adjustable couplings. Optionally, the method can include conforming the first and second occipital plates to an anatomical location adjacent the foramen magnum and offset from an axis defined by the spinal column.

These aspects, as well as others, will now be described in detail.

### BRIEF DESCRIPTION OF THE DRAWINGS

The devices and methods provided herein will be more fully understood from the following detailed description taken in conjunction with the accompanying drawings, in which:

FIG. 1 is a perspective view of a prior art spinal fixation system;

FIG. 2A is a perspective view of an embodiment of first and a second spinal fixation devices engaged at desired anatomical locations;

FIG. 2B is an alternative view of the spinal fixation devices of FIG. 2A;

FIG. 3A is a perspective view of an exemplary embodiment of a spinal fixation device;

FIG. 3B is an exploded view of the device of FIG. 3A;

FIG. 4A is a perspective view of another exemplary embodiment of an occipital plate;

FIG. 4B is a perspective view of another exemplary embodiment of an occipital plate;

FIG. 5A is another exemplary embodiment of a spinal fixation device; and

FIG. 5B is an exploded view of the device of FIG. 4A.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope is defined solely by the claims. The features illustrated or described in connection with one exemplary embodiment may be combined with the features of other embodiments. Such modifications and variations are intended to be included within the scope of the present disclosure.

Devices, systems, and methods for optimizing various cervical stabilization procedures are described herein. As summarized above, the presently disclosed embodiments provide a surgeon with the ability to engage a fixation element to an optimal location of the patient's anatomy without being limited to a location along the midline of a patient's spinal column and/or without being limited with respect to the exact orientation of a fixation element(s) (e.g., the fixation rod) to be engaged by the fixation plate. More specifically, the fixation plates provided herein include an elongate, generally planar fixation plate which can be configured for placement at any desired anatomical location. In an exemplary embodiment, the plate can be configured so as to include a series of bone screw receiving thru-hole(s) positioned proximate a center-line of the elongate plate. For example, the thru-holes can be positioned along the center-line or the thru-holes can be positioned such that at least one thru-hole is off-set from the center line. Additionally, the plate can include a position-adjustable coupling element configured to engage a fixation element wherein the coupling element can also be positioned proximate the center-line. As will be shown, the presently disclosed devices and methods allow for increased versatility in that the coupling element for the fixation element can be rotatable, translatable, and/or capable of polyaxial movement relative to the elongate member thus allowing the coupling element to be easily aligned with a superior end of a fixation element. Such versatility allows the fixation device to be positioned independent of the exact orientation of the fixation rod. In light of these various features, the devices and methods provided herein allow a surgeon to engage a fixation plate at an optimal location of the occipital bone, position a spinal fixation element along a series of vertebrae, manipulate a coupling element so as to align the coupling element with the superior end of the fixation element, and securely engage the fixation element to the coupling element.

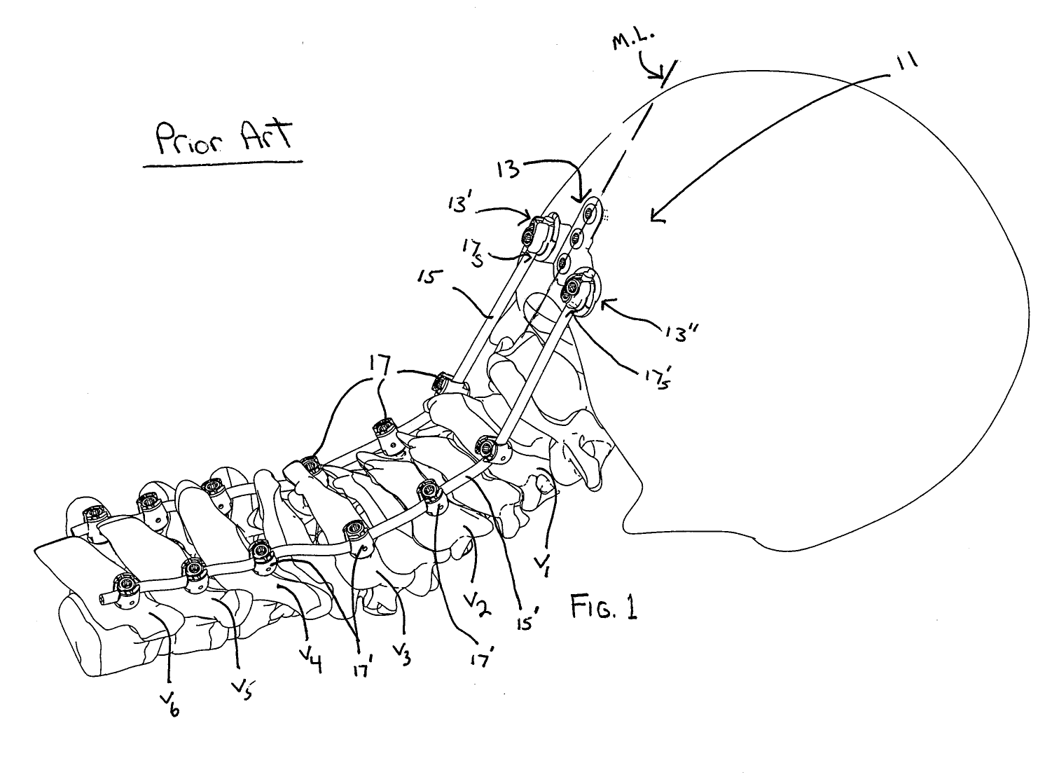

As indicated above, the presently disclosed devices and methods provide numerous advantages over traditional spinal stabilization techniques. For example, FIG. 1 shows a commonly used technique in which a prior art occipital plate 13 is fixed along the midline (M.L.) of a patient's occipital bone 11. As shown, the procedure typically requires a first plurality of fixation assemblies 17 (e.g., a bone-anchor coupled to a receiving head) engaged to a plurality of vertebrae V₁, V₂, V₃, V₄, V₅, V₆ along one side of the midline (M.L.) of the patient's spinal column, and a second plurality of fixation assemblies 17′ along an opposite side of the midline (M.L.). Once the fixation assemblies 17, 17′ are positioned as such, a first fixation element 15 can be engaged to the first plurality of fixation assemblies 17, and a second fixation element 15′ can be engaged to the second plurality of fixation assemblies 17′. Next, a superior portion 17s, 17s′ of each fixation assembly 17, 17′ can be engaged to various coupling elements 13′, 13″ fixed to the occipital plate 13. Using such instrumentation, the surgeon is required to position the plate 13 along the midline (M.L.) of the occipital bone 11 in order to ensure that both the first and second fixation elements 15, 15′ can be engaged to the same plate 13. A drawback to such an approach is that the surgeon cannot select the optimal bone location for each fixation assembly, and must instead utilize a location accessible to both fixation elements 15, 15′.

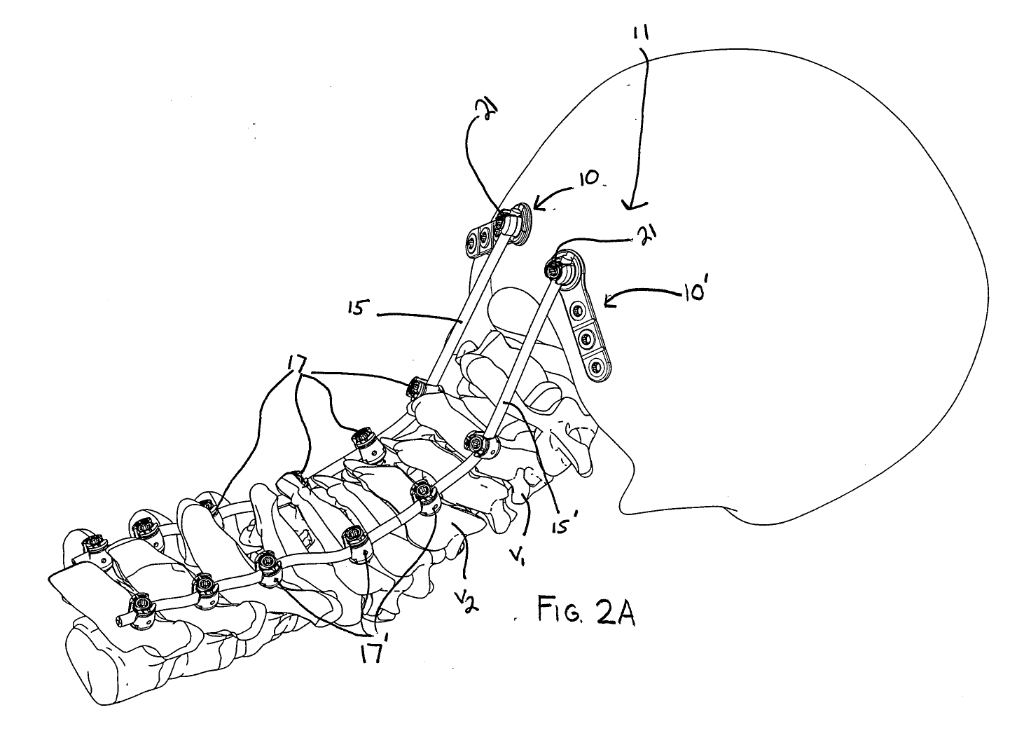

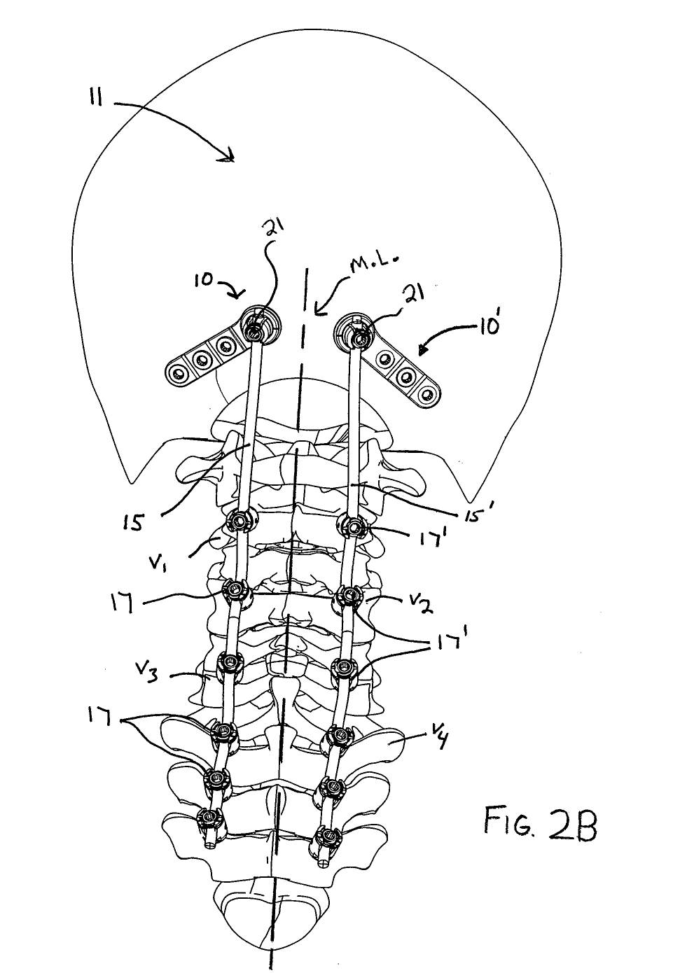

In contrast, the presently disclosed devices, systems, and methods enable a surgeon to position a customized occipital plate at a location deemed optimal for a desired procedure (e.g., where there is a sufficient amount of healthy bone mass). For example, FIGS. 2A-2B illustrate a stabilization procedure performed with exemplary embodiments of the presently disclosed occipital plate(s) 10, 10′. As shown, the occipital plates 10, 10′ can be engaged to the patient's occipital bone 11 in any position and/or in any orientation as desired by the surgeon. For example, each occipital plate 10 can be configured as an elongate plate capable of conforming to an anatomical location adjacent the midline (M.L) of a patient's occipital bone 11. Further, since each occipital plate 10, 10′ is configured to releasably engage only a single fixation element 15, 15′, the plates 10, 10′ can be engaged to the occipital bone 11 independent of one another thereby adding to the versatility of the procedure. As will be described below, the ability to independently position the plates 10, 10′ adjacent the midline (M.L.) of the patient's spinal column provides significant advantages to those procedures where the patient has an oversized foramen magnum which renders it impossible for the surgeon to position any type of plate along the midline (M.L.) of the patient's occipital bone 11.

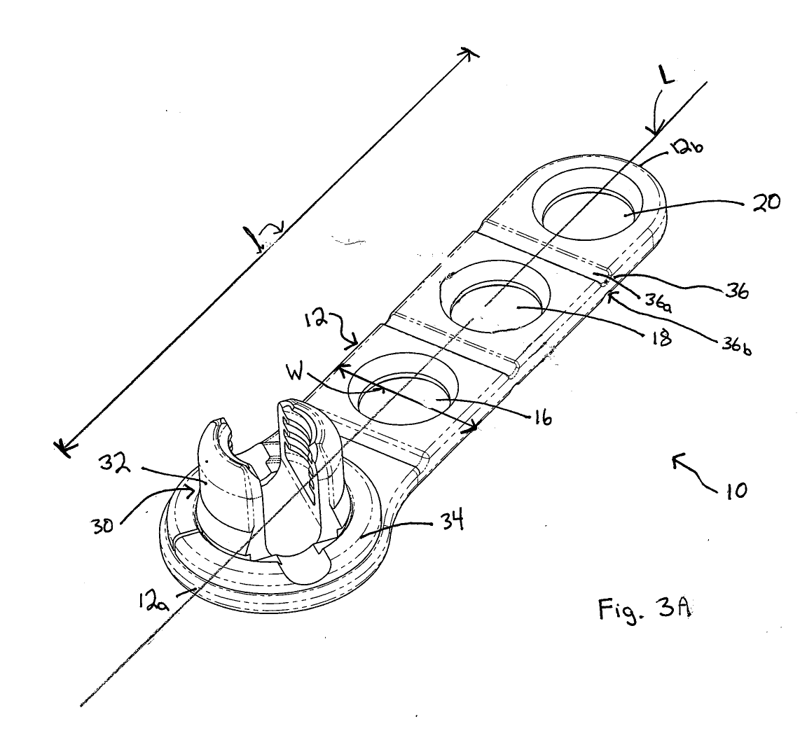

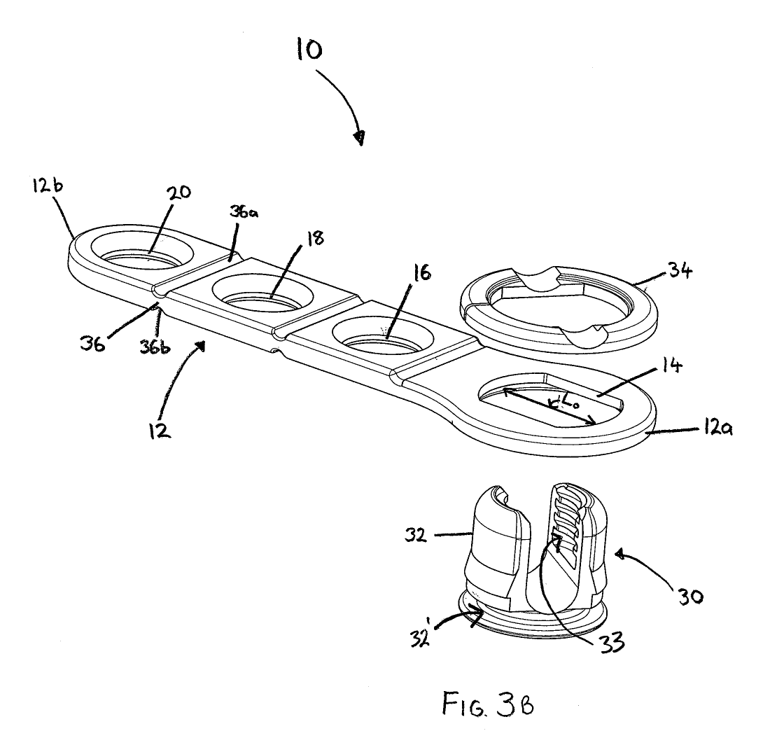

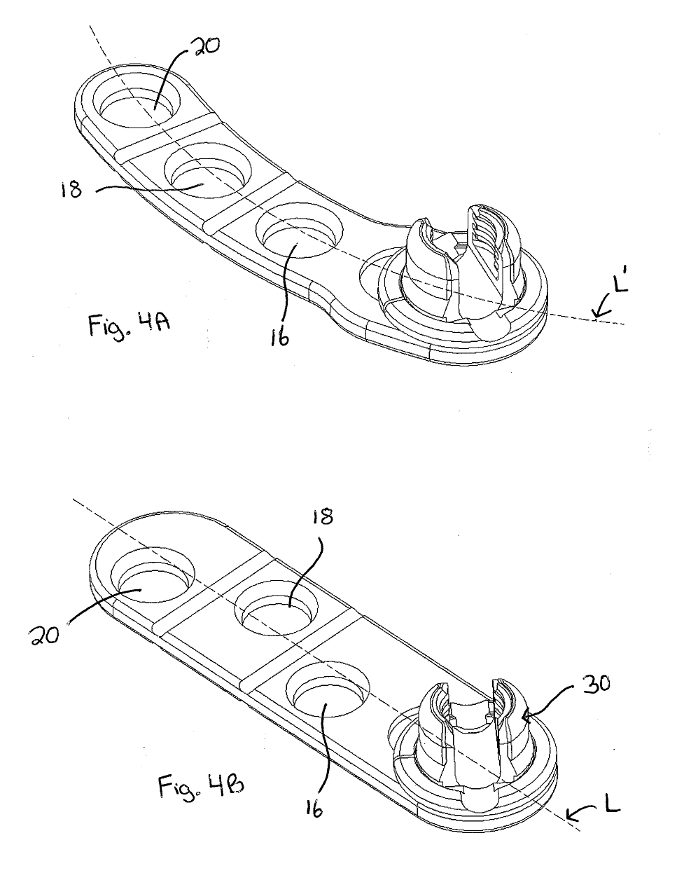

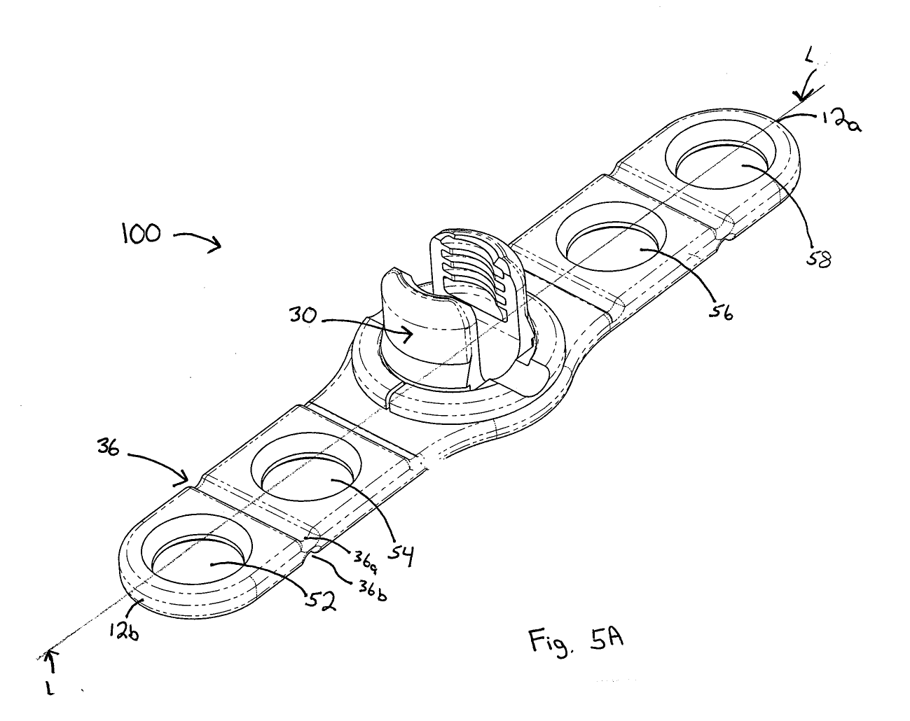

FIGS. 3A-3B provide an exemplary embodiment of the presently disclosed in-line occipital plate 10. As shown, the occipital plate 10 can include a generally elongate member 12 that defines a center-line (L) extending between inferior and superior ends 12a, 12b thereof. As shown in FIG. 4A, the center line (L′) can also be curved. The shape of the elongate member 12 can vary, but in an exemplary embodiment the elongate member 12 can be substantially planar wherein the inferior and superior ends 12a, 12b have a rounded or convex profile to avoid the risk of damage during implantation. In other embodiments, as shown in FIG. 4A, the elongate member can be curved. Although the plate 10 can be generally planar in an initial configuration, it is understood that a surgeon can contour the plate to conform to the area of implantation. Alternatively, the plate 10 may be contoured. The length (l) and width (w) of the elongate member 12 can also vary, and will typically depend on the nature of the procedure and/or the patient's anatomy. For example, in one embodiment, the elongate member 12 can have a substantially constant width (w) from the first end 12a to the second end 12b of the plate 10. In an exemplary embodiment, the length (l) of the plate 10 can range from about 30 mm to about 50 mm, and the width (w) of the plate 10 can range from about 8 mm to about 15 mm.

The occipital plate 10 can also include any number (e.g., 1, 2, 3, 4, 5, etc.) of bone screw receiving thru-holes configured to receive a corresponding number of bone screws (not shown) or any another type of suitable anchoring devices so as to anchor the plate 10 to the underlying occipital bone 11. For example, the exemplary embodiment of FIG. 3A includes three such bone screw receiving thru-holes 16, 18, 20. As will be apparent to those skilled in the art, the bone-screw receiving thru-holes 16, 18, 20 can be of any shape (e.g., circular, oval, etc.) and/or diameter capable of securely receiving the bone screw or other suitable anchoring device. Also, the thru-holes 16, 18, 20 can be substantially similar in shape (as shown) or they can each have a distinct shape(s) and/or diameter(s). The alignment and/or positioning of the thru-holes 16, 18, 20 relative to the elongate member 12 can also be optimized to conform to the desired anatomical location. In an exemplary embodiment, the thru-holes 16, 18, 20 can be substantially aligned or positioned proximate along the center line (L) of the elongate member 12 thereby providing optimal stability for positioning of the occipital plate 12 at a location adjacent the midline of the patient's spine. For example, as shown in FIG. 3A, the thru-holes 16, 18, 20 can be aligned along the center-line (L). Alternatively, as shown in FIG. 4B, the thru-holes 16, 18, 20 (or at least one thereof) can be positioned offset from the center-line. In such an embodiment, the holes 16, 18, 20 can be staggered along a length of the plate. Additionally, as will be described in detail below, the occipital plate 10 can include a coupling element 30 capable of releasably engaging a fixation element (15, see FIGS. 2A-2B). The coupling element 30 can also be positioned proximate the center-line (L) of the plate 10, further optimizing the plate's ability to conform to a location adjacent the midline of the spinal column.

As noted above, the various embodiments of the presently disclosed occipital plate 10 include a position-adjustable coupling element 30 configured to releasably engage a single fixation element thereby providing several advantages over commonly used devices. More specifically, the ability to engage only a single fixation element allows the surgeon to engage the occipital plate 10 to an anatomical location without concern as to the relative positioning of a second fixation element. Also, the ability to releasably engage the fixation element allows the surgeon to first select the optimal location and then securely engage the fixation element thereto. Thus, the surgeon is not restrained by finding a location which accommodates the fixation element already engaged to the occipital plate. Additionally, as will also be detailed below, the coupling element 30 can be configured to be rotatable, translatable, and/or capable of polyaxial movement relative to the elongate member 12 of the plate thereby facilitating the surgeon's ability to engage the fixation element to the coupling element 30. These various advantages are now described in detail.

Referring to FIGS. 3A-3B, the coupling element 30 can be any element capable of releasably engaging a spinal fixation element to the elongate member 12. More specifically, the coupling element 30 can be a cylinder-like object 32 having a U-shaped opening formed therein which is configured to receive the fixation element. In an exemplary embodiment, the coupling element 30 can be a slotted bolt. By way of non-limiting example, U.S. Pat. No. 6,524,315 of Selvitelli et al. entitled “Orthopaedic Rod/Plate Locking Mechanism,” and U.S. Pat. No. 6,547,790 of Harkey, III et al. entitled “Orthopaedic Rod/Plate Locking Mechanism and Surgical Methods,” the entirety of these references being incorporated by reference herein, each describe various examples of features of coupling elements 30 that can be utilized with the presently disclosed occipital plate 10.

The coupling element 30 can be engaged to and/or formed on the elongate member 12 at a location proximate (e.g., in alignment with or offset from) the center-line in any number of ways. For example, as indicated by the exploded view of FIG. 3B, the coupling element 30 can be secured to the elongate member 12 via a thru-hole 14 formed in the member 14. After the coupling element 30 is positioned within the thru-hole, an engagement ring 34 can be placed over the coupling element 30 and secured to the element 30 via a groove 32′ formed therein. Once secured as such, a fixation element can be positioned within the U-shaped opening and secured using a set screw 21 (FIGS. 2A and 2B) or any other suitable closure element. As shown, the inner portion of the U-shaped opening can include a series of threads 33 adapted to engage a corresponding series of threads (not shown) formed in the set screw 21 thereby securing the fixation element within the coupling element 30.

As an added advantage, the coupling element 30 can be manipulated relative to the elongate member 12 in various ways thereby facilitating engagement of the fixation element thereto. More specifically, the ability to manipulate the coupling element 30 relative to the elongate member 12 allows the surgeon to engage the plate 10 to an optimal anatomical location and then further manipulate the coupling element 30 so as to align the coupling element 30 with the fixation element 15 (see FIGS. 2A and 2B). Manipulation of the position adjustable coupling element 30 relative to the elongate member 12 can allow for various ranges of motion of the element 30 relative to the plate 12. For instance, the coupling element 30 can be configured to be rotatable relative to the elongate member 12 thereby allowing the U-shaped opening to be rotated after the elongate plate 12 is engaged to the occipital bone 11. Various embodiments can allow for various degrees of rotation. For example, the coupling element 30 can be configured to rotate in only one direction (e.g., clockwise) or both directions (clockwise and counter-clockwise). Additionally, the coupling element 30 can be configured to rotate a limited amount (e.g., about 45 degrees) or the coupling element 30 can be configured to rotate 360 degrees. Those skilled in the art will appreciate that the coupling element 30 can be engaged to the elongate member 12 in a variety of manners so as to provide the desired rotation. For example, as shown in FIGS. 3A and 3B, the substantially cylindrical shape of the coupling element 30 can allow for the coupling element 30 to rotate relative to the opening 14 of the elongate member 12.

In other embodiments, the coupling element 30 can be configured to be translatable relative to the elongate member 12. The ability to translate the coupling element 30 along the elongate member 12 further facilitates the surgeon's ability to align the coupling element 30 with the fixation element. As will be apparent to those skilled in the art, the coupling element 30 can be engaged to the elongate member 12 in any number of ways so as to provide such translatable movement. For example, as shown in FIG. 3B, the coupling element 30 can be disposed within an elongated thru-hole 14 thereby allowing the coupling element 30 to move laterally along the opening 14. As will also be apparent to those skilled in the art, the length of the opening (Lₒ) can vary depending on the requirements of the procedure and/or the patient's anatomy.

In other embodiments, the coupling element 30 can also be configured to be capable of polyaxial movement relative to the elongate member 12. Those skilled in the art will appreciate that the coupling element 30 can be engaged to the elongate member 12 in various ways to provide such polyaxial movement. For example, as shown in FIG. 3B, the groove 32′ of the coupling element 30 can have a semi-cylindrical shape. Additionally, a bottom portion of the groove 32′, the top and bottom surfaces of the elongate member 12, and a bottom portion of the washer 34 can also include mating cylindrical surfaces thereby allowing for polyaxial movement when the element 30 is coupled to the opening 14 of the elongate member 12.

In some embodiments, the occipital plate 10 can be configured to adapt and/or conform to a target anatomical location (e.g., adjacent the midline of a patient's spinal column). As will be apparent to those skilled in the art, the occipital plate 10 can be configured as such in a variety of manners. For example, the occipital plate 10 can be formed of a flexible or malleable material thereby allowing for the plate 10 to bend and accommodate the target anatomical location. In other embodiments, referring again to FIGS. 3A-3B, the spinal fixation plate 10 can include at least one bend zone 36 formed therein for allowing the elongate member 12 to conform the plate to a surface of the target anatomical location. As shown, the bend zones 36 can be formed from grooves or channels that extend across at least one of the front surface 36a or the back surface 36b of the elongate member 12. Those skilled in the art will appreciate that a variety of other techniques can be used to provide bendable movement of one or more portions of the spinal fixation plate 10, and that the bend zones can be formed at any location along the elongate member 12.

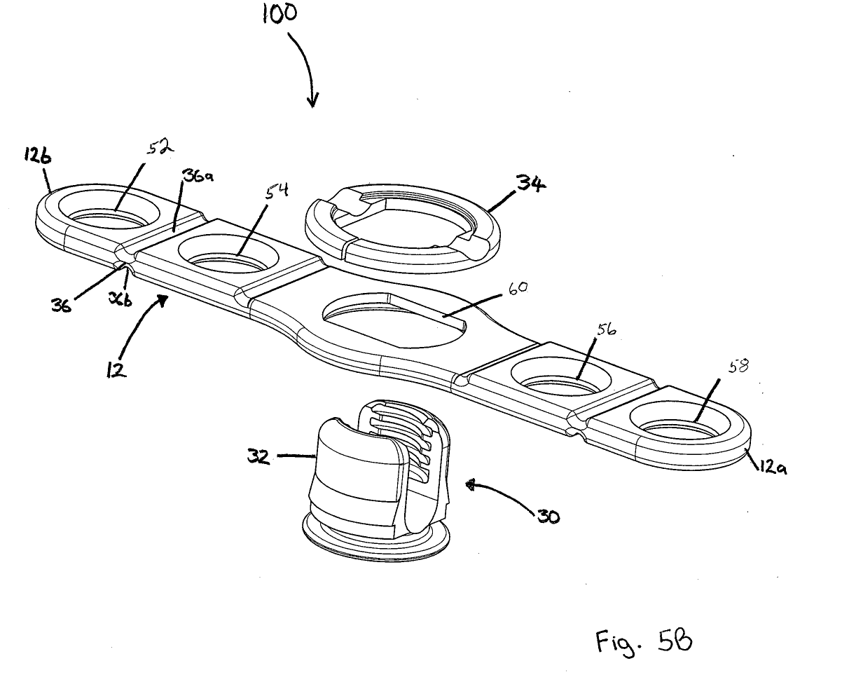

In addition to the embodiments described above, the configuration of the occipital plate 10 can vary depending upon the needs of a particular surgical procedure. For example, FIGS. 5A-5B provide another exemplary embodiment of the fixation plate 100 in which the coupling element 30 is positioned substantially in the middle of the elongate member 12. Also, as shown, the fixation plate 100 can include a first plurality of bone screw receiving thru-holes 52, 54 positioned on one side of the coupling element 30 and additional bone-screw receiving thru-holes 56, 58 on the opposite side of the coupling element 30. Like the previously described embodiments, the thru-holes 52, 54, 56, 58 and the coupling element 30 can all be positioned proximate the center-line (L) of the elongate member 12 of the plate 100. Such a configuration can allow the plate 100 to be positioned adjacent the midline (M.L.) of the patient's spinal column. As shown in the exploded view of FIG. 5B, and similar to the embodiment described above, the coupling element 30 can be engaged to the fixation plate 100 via a thru-hole 60 disposed in the plate 100. As described above, the coupling element 30 can be configured to be rotatable, translatable, and/or configured for polyaxial movement relative to the elongate member 12. In other embodiments, the occipital plate can include any number and/or orientation of thru-hole(s) and/or the coupling element can be positioned at any location relative to the elongate member 12 (e.g., middle, inferior end, etc.).

In addition to the various embodiments of the spinal fixation plate described above, methods are also provided herein for occipital coupling of a spinal fixation element. In an exemplary embodiment, the method includes fixing an inferior portion of a spinal fixation element to one or more vertebrae and further providing an occipital plate of the types described above and illustrated in FIGS. 3A-5B capable of releasably engaging a portion of the fixation element.

The method further includes fixing the occipital plate to a desired anatomical location. In an exemplary embodiment, such a desired anatomical location is a location adjacent the foramen magnum and offset from an axis defined by the spinal column where there is a sufficient quantity of healthy bone in which to anchor the plate. As explained above, positioning the plate adjacent the foramen magnum is particularly useful in those procedures where the patient has an enlarged foramen magnum. Once a desired anatomical location has been selected, the method can further include engaging the fixation element to the coupling element. As shown in FIGS. 2A-2B, the method can further include positioning a first fixation plate 10 along one side of the midline of the patient's spine, and positioning a second fixation plate 10′ on the opposite side of the midline of the patient's spinal column thereby allowing for first and second fixation elements 15, 15′ to be positioned on opposite sides on the midline of the spinal column.

A person skilled in the art will appreciate that the various methods, systems, and devices disclosed herein can be formed from a variety of materials. Moreover, particular components can be implantable and in such embodiments the components can be formed from various biocompatible materials known in the art. Exemplary biocompatible materials include, by way of non-limiting example, composite materials, polymeric materials, biocompatible metals and alloys such as stainless steel, titanium, titanium alloys and cobalt-chromium alloys, and any other material that is biologically compatible and non-toxic to the human body.

One skilled in the art will appreciate further features and advantages based on the above-described embodiments. Accordingly, the disclosure is not to be limited by what has been particularly shown and described, except as indicated by the appended claims. All publications and references cited herein are expressly incorporated herein by reference in their entirety.

What is claimed is:

1. An implantable spinal fixation device, comprising:
an elongate member having a first end and a second end with a center-line extending therebetween;
a plurality of bone screw receiving thru-holes formed in the elongate member, the thru-holes positioned proximate the center-line of the elongate member; and
a position-adjustable coupling element engaged to a portion of the elongate member, the coupling element positioned proximate the center-line and configured to releasably engage a spinal fixation element.

2. The device of claim 1, wherein the plurality of thru-holes are positioned along the center-line of the elongate member.

3. The device of claim 1, wherein at least one thru-hole is positioned offset from the center line.

4. The device of claim 1, wherein the center-line is a straight line.

5. The device of claim 1, wherein the center-line is curved.

6. The device of claim 1, wherein the coupling element is rotatable relative to the elongate member.

7. The device of claim 1, wherein the coupling element is translatable along the center-line of the elongate member.

8. The device of claim 1, wherein the coupling element is configured for polyaxial motion relative to the elongate member.

9. The device of claim 1, wherein at least one of the plurality of thru-holes is elongated.

10. The device of claim 9, wherein the coupling element is configured to be secured to the elongated thru-hole.

11. The device of claim 10, wherein the thru-hole configured to engage the coupling element is positioned in a central portion of the elongate member.

12. The device of claim 10, wherein the thru-hole configured to engage the coupling element is positioned at an inferior portion of the elongate member.

13. An in-line occipital plate, comprising:
an elongate plate member having a center-line extending from a first end of the member to a second end of the member, the elongate plate member being conformable to an anatomical location,
wherein the elongate plate member includes a single position-adjustable coupling element configured to releasably engage a single spinal fixation element, and the elongate plate member further includes at least one bone screw receiving thru-hole, the position-adjustable coupling element and the at least one bone screw receiving thru-hole being positioned proximate the center-line of the elongate member.

14. The device of claim 13, wherein the coupling element is translatable along the center-line of the elongate member.

15. The device of claim 13, wherein the coupling element is positioned in a central portion of the elongate member.

16. The device of claim 13, wherein the coupling element is positioned at an inferior portion of the elongate member.

17. The device of claim 13, wherein the coupling element is configured for polyaxial motion relative to the elongate member.

18. A system, comprising:
an occipital plate engaged to an occiput, the occipital plate comprising:
an elongate member having a first end and a second end with a center-line extending therebetween;
a plurality of bone screw receiving thru-holes formed in the elongate member, the thru-holes positioned proximate the center-line of the elongate member; and
a position-adjustable coupling element engaged to a portion of the elongate member, the coupling element positioned proximate the center-line and configured to releasably engage a spinal fixation element;
a bone anchor implanted in a vertebra; and
a spinal fixation element connecting the bone anchor and the occipital plate.

19. The system of claim 18, wherein the coupling element of the occipital plate is rotatable relative to the elongate member.

20. A method of occipital coupling of a spinal fixation element, comprising:
fixing an inferior portion of a spinal fixation element to one or more vertebra;
providing an occipital plate having a superior end and an inferior end and a plurality of bone screw receiving thru-holes positioned proximate a center-line of the occipital plate, the occipical plate further including a position-adjustable coupling element positioned proximate the center-line of the occipital plate;
fixing the occipital plate adjacent the foramen magnum and offset from a midline defined by the spinal column; and
fixing a superior portion of the spinal fixation element to the position-adjustable coupling element.

21. The method of claim 20, further comprising:
fixing an inferior portion of a second spinal fixation element to one or more vertebra;
providing a second occipital plate having a superior end and an inferior end and a plurality of bone screw receiving thru-holes positioned proximate a center-line of the second occipital plate, the second occipital plate further having a position-adjustable coupling element positioned proximate the center-line of the second occipital plate;
fixing the second occipital plate adjacent the foramen magnum and offset from the midline defined by the spinal column; and
fixing a superior portion of the second spinal fixation element to the position-adjustable coupling of the second occipital plate.

22. The method of claim 20, further comprising:
translating the position-adjustable coupling element relative to the occipital plate so as to align the coupling with the superior portion of the spinal fixation element.

23. The method of claim 20, further comprising:
polyaxially adjusting the position-adjustable coupling element relative to the occipital plate so as to align the coupling with the superior portion of the spinal fixation element.

24. A method of occipital coupling of a spinal fixation element, comprising:
providing first and second spinal fixation elements;
fixing an inferior portion of the first and second spinal fixation elements to one or more vertebra, the first and second spinal fixation elements positioned on opposites sides of a midline defined by a patient's spinal column;
providing a first and a second occipital plate, each occipital plate having a first end, a second end and a center-line extending therebetween, each plate further having a plurality of thru-holes proximate respective center-lines, and each plate also having a single rotatable and translatable coupling element positioned within an elongated thru-hole;
fixing the first occipital plate adjacent the foramen magnum and offset in a first lateral direction from the midline defined by the spinal column;
fixing the second occipital plate adjacent the foramen magnum and offset in a second lateral direction from the midline defined by the spinal column, the first and second occipital plates positioned on opposite sides of the midline defined by the spinal column;
manipulating the coupling elements relative to the first and second occipital plates so as to align the first coupling with the superior portion of the first spinal fixation element and the second coupling with the superior portion of the second spinal fixation element; and
fixing the superior portion of the first fixation element to the first rotatable coupling and fixing the superior portion of the second spinal fixation element to the second rotatable coupling.

25. The method of claim 24, further comprising:
conforming the first occipital plate to a first anatomical location adjacent the foramen magnum and offset from the midline defined by the spinal column; and
conforming the second occipital plate to a second anatomical location adjacent the foramen magnum and offset from the midline defined by the spinal column, the first and second anatomical location positioned on opposite sides of the midline defined by the patient's spinal column.
